(19) United States
(12) Patent Application Publication
(10) Pub. No.: US 2006/0148740 A1
(43) Pub. Date: Jul. 6, 2006

(54) Mannose-6-phosphate receptor mediated gene transfer into muscle cells

(75) Inventor: Gerard Johannes Platenburg, Voorschoten (NL)

Correspondence Address: FITCH, EVEN, TABIN & FLANNERY, P. O. BOX 65973, WASHINGTON, DC, 20035, US

(73) Assignee: Prosensa B.V., Leiden (NL)

(21) Appl. No.: 11/028,574

(22) Filed: Jan. 5, 2005

(51) Int. Cl. A61K 48/00

(52) U.S. Cl. 514/44

(57) ABSTRACT

The invention relates to glycoside-compound conjugates for use in antisense strategies and/or gene therapy. The conjugates comprise a glycoside linked to a compound, in which the glycoside is a ligand capable of binding to a mannose-6-phosphate receptor of a muscle cell. For example the cells are muscle cells of a Duchenne Muscular Dystrophy (DMD) patient and the conjugate comprises an antisense oligonucleotide which causes exon skipping and induces or restores the synthesis of dystrophin or variants thereof.

### FIELD OF INVENTION

The present invention is in the field of glycoside conjugates. It relates to improving muscle uptake of compounds in general. In particular it relates to glycoside-oligonucleotide conjugates for use in antisense strategies and gene therapy.

### BACKGROUND OF THE INVENTION

A potential genetic therapy was explored, aimed at restoring the reading frame in muscle cells from Duchenne muscular dystrophy (DMD) patients through targeted modulation of dystrophin pre-mRNA splicing. Considering that exon 45 is the single most frequently deleted exon in DMD, whereas exon (45+46) deletions cause only a mild form of Becker muscular dystrophy (BMD), an antisense-based system was set up to induce exon 46 skipping from the transcript in cultured myotubes of mouse and of human origin. In myotube cultures from two unrelated DMD patients carrying an exon 45 deletion, the induced skipping of exon 46 in only 15% of the mRNA led to normal amounts of properly localized dystrophin (of course lacking the domains corresponding to exon 45 & 46) in at least 75% of myotubes (van Deutekom et al. 2001). Using the same antisense-based strategy using a different antisense sequence, in another study the skipping of 11 other exons was demonstrated in the dystrophin gene in cultured human myotubes (Aartsma-Rus et al. 2002). Technology to induce skipping of these 12 different exons would (in the population of DMD causing genetic defects), in total, allow correction of more than 50% of the deletions and 22% of the duplications in the population present in the Leiden DMD-mutation Database.

However, the biggest hurdle to overcome is the poor in vivo muscle uptake of these antisense oligonucleotides, and this applies for other molecules with therapeutic potential as well, by the relevant cells. An efficient therapy for DMD will require that essentially all of the skeletal muscles including those of arms and legs and the muscles involved in respiration as well as the cardiac muscle are targeted. None of the mechanisms investigated to date have the ability to specifically deliver (antisense) oligonucleotides, let alone entire genes, to essentially all muscle tissues/cells simultaneously over the entire body. Methods for the in vivo delivery of genes or other compounds into muscle that have been published so far include injection of naked DNA with or without electrotransfer, intravascular delivery (both reviewed in Herweijer and Wolff, 2003) and use of microbubbles (Lu et al. 2003). Direct injection of DNA into the skeletal muscle is a safe and simple method, but is hampered by low transfection efficiencies. The efficiencies can be significantly improved by pretreatment of the muscle with hyaluronidase followed by electrotransfer and using this method a dystrophin plasmid was expressed in 22% of the fibres in the muscle of an mdx mouse for up to 8 weeks (Gollins et al. 2003). A severe obstacle to clinical application of this method however, is the muscle fibre damage induced by the powerful electric fields required to achieve efficient gene delivery. A way to limit the damage to the muscles, is injection into skeletal muscle of a mixture of naked DNA and microbubbles. It was found that the use of a commercially available albumin-coated octa-fluoropropane gas microbubble, Optison, improves transfection efficiency and this was associated with a significant decrease in muscle damage (Lu et al. 2003). However, the major disadvantage of direct injection into muscles remains, being that each muscle has to be treated separately, and thus treatment of the entire muscle mass of an individual by these methods is not feasible.

The intravascular delivery of DNA is a more attractive method, because a whole muscle group can be covered with a single injection. Intravascular delivery via a catheter to limb skeletal muscle groups, in combination with blocking blood flow with a blood pressure cuff, has successfully been performed in rabbits, dogs and rhesus monkeys (Herweijer and Wolff, 2003). In rhesus monkeys, transfection efficiencies ranging from less than 1% to more than 30% in different muscles in leg and arm have been observed (Zhang et al. 2001). Also, it is claimed that delivery is not limited to skeletal muscle, but that delivery is also in the cardiac muscle (Herweijer et al., 2000). However, whole-body treatment would still require multiple injections and furthermore, treatment of the respiratory muscles seems impossible with this method.

Ideally, whole-body muscle therapy would use single intravenous injections of a compound endowed with a cell specific targeting ability. Up to date, two molecules have been described that have potential for muscle cell targeting. The first is a peptide sequence with enhanced in vivo skeletal and cardiac muscle binding, that was identified by screening a random phage display library (Samoylova and Smith, 1999). Muscle selectivity of the phage clone carrying this peptide was estimated to be in the range of 9- to 20-fold for skeletal and 5- to 9-fold for cardiac muscle (depending on control tissue) as compared to phage with no insert. However, it has not yet been shown whether or not this peptide can be used for in vivo targeting of conjugated compounds to muscle cells. The other molecule that has been described is an Fv part of a monoclonal antibody (mAb) that is selectively transported into skeletal muscle in vivo (Weisbart et al. 2003). Single chain Fv fragments of the murine mAb were injected into the tail veins of mice and 4 hours later the fragments were found in 20% of skeletal muscle cells, primarily localized in the nucleus. It was shown that the mAb binds to the protein myosin IIb in lysates of skeletal muscle cells, but it did not bind any protein in lysates of heart muscle cells. Therefore, this antibody might be useful for targeting to skeletal muscles, but not to the heart muscle.

Mannose-6-phosphate (M6P) residues are uniquely recognized by the two members of the P-type lectin family, the ˜46-kDa cation dependent mannose-6-phosphate receptor (CD-MPR) and the ˜300 kDa insulin-like growth factor II/mannose-6-phosphate receptor (IGF-II/MPR) (Dahms and Hancock, 2002). The P-type lectins play an essential role in the generation of functional lysosomes within the cells of higher eukaryotes, by directing newly synthesized lysosomal enzymes bearing the M6P signal to lysosomes. Lysosomal enzymes are synthesized by membrane bound ribosomes and translocated to the endoplasmic reticulum (ER), where the nascent proteins are glycosylated with high-mannose oligosaccharide chains. The mannose residues are then phosphorylated during further transit of the proteins through the ER-Golgi biosynthetic pathway, generating the M6P ligand used in targeting of the lysosomal enzymes to the lysosome via the M6P-receptors (Dahms and Hancock, 2002). At the cell surface the IGF-II/MPR, but not the CD-MPR, binds and internalizes a diverse population of M6P-containing proteins and is responsible for endocytosis of the majority of extracellular lysosomal enzymes (Ghosh et al. 2003; Hassan, 2003). The IGF-II/MPR is present in several human tissues such as kidney, liver, spleen and lung and also in heart and skeletal muscle (Funk et al. 1992; Wenk et al. 1991), and can therefore be used for targeting and uptake of M6P-containing compounds into the lysosomal compartment of muscle cells. The feasibility of such an approach has been demonstrated with the lysosomal enzyme α-glucosidase (GAA). First of all, it was shown that GAA isolated from bovine testis was endocytosed in a M6P-receptor dependent manner by cultured human skeletal muscle cells, obtained from muscle biopsies (Reuser et al. 1984). The uptake could completely be inhibited by M6P and by bovine testis β-galactosidase, a lysosomal enzyme bearing phosphorylated high-mannose-type sugar chains. These results show that M6P-receptors are present on the plasma membrane of skeletal muscle cells and engaged in the uptake of M6P-containing lysosomal enzymes. Also, when recombinant human GAA (rhGAA), produced in CHO-cells or mouse milk, was added to human GAA -/- fibroblasts in cell culture, the enzyme was internalized in a M6P-receptor dependent manner (Bijvoet et al. 1998; Martiniuk et al. 2000). Finally, after injection of rhGAA into GAA knockout mice, uptake into heart, skeletal muscles, legs and respiratory muscles, among which diaphragm, was demonstrated (Bijvoet et al. 1998; Martiniuk et al. 2000).

### DISCLOSURE OF THE INVENTION

The present invention provides a novel method of delivering compounds into extra-lysosomal compartments, such as the cytoplasm, ER and the nucleus, of cells, in particular muscle cells. It was unexpectedly found that conjugates comprising a glycoside, such as the monosaccharide M6P, were able to deliver compounds linked to said monosaccharide into the nucleus of muscle cells, despite the prior art teaching that M6P is specifically targeted to the lysosomal compartment of cells. This finding is of particular benefit in antisense strategies and/or gene therapy that involve the delivery of functional moieties to, or moieties that are functionalized in the nucleus.

In one embodiment of the invention glycoside-compound conjugates are provided. A “conjugate” as used herein refers to a ligand, such as a glycoside, which is chemically conjugated to a compound of interest. The ligand is able to bind to a specific receptor and thereby directs (or targets) the conjugate to this receptor. In one embodiment of the invention the ligand is capable of binding to an M6P receptor, preferably to IGF-II/MPR. Preferably the M6P receptor is of a muscle cell. The glycoside is preferably a mono-, di-, tri- or a higher order saccharide. In a preferred embodiment the saccharide is a M6P residue, although other saccharides with binding specificity for muscle cell receptors can be used. The conjugate may comprise one, two, three, four or more glycosides. For example, the conjugate may comprise (M6P)₂ or (M6P)₄ or additional M6P residues. In case the conjugate comprises more than one glycoside it is preferred the terminal glycoside is an M6P.

Thus the invention relates to a conjugate comprising a glycoside linked to a compound in which said glycoside is a ligand capable of binding to a mannose-6-phosphate receptor of a cell having such a receptor. In particular the invention relates to a conjugate comprising a glycoside linked to a compound in which said glycoside is a ligand capable of binding to a mannose-6-phosphate receptor of a muscle cell. In an embodiment said compound is an oligonucleotide or oligonucleotide equivalent, such as an RNA, DNA, Peptide Nucleic Acid (PNA) or Locked Nucleic Acid (LNA). In an embodiment said oligonucleotide or oligonucleotide equivalent is in antisense orientation. In an embodiment said oligonucleotide or oligonucleotide equivalent comprises at least 10 nucleotides identical to or complementary to a human dystrophin gene. In an embodiment said oligonucleotide or oligonucleotide equivalent is selected from one of the following: morpholino, 2′-O-methyl RNA and 2′-O-allyl RNA. U.S. Pat. No. 6,172,208 discloses an oligonucleotide wherein at least one nucleotide unit is conjugated with a sugar or sugar phosphate. In particular for an oligonucleotide equivalent such as PNA or LNA a length equivalent to at least 10 nucleotides or even 9 or 8 may be sufficient. For RNA and DNA oligonucleotides a length of more than 10, e.g. at least 11, 12, 13, 14, 15, 16, 17, 18, 19, 20 or more than 20 nucleotides may be beneficial. Usually oligonucleotides need not be longer than about 25 nucleotides in length.

In a preferred embodiment said mannose-6-phosphate receptor is an insulin-like growth factor II/mannose-6-phosphate receptor (IGF-II/MPR).

It is to be understood that in the context of this invention ‘a glycoside linked to an oligonucleotide’ includes non-covalent linkage of the nucleotide via a cationic oligonucleotide complexing species such as known cationic transfection promoting agents such as spermine and in particular polyethyleneimine (PEI). For example in the conjugate of the invention the glycoside is covalently coupled to PEI and the oligonucleotide is complexed to the PEI via non-covalent interactions. Such an approach is of particular interest for delivery of larger polynucleotides including genes and expression sequences therefor. Thus a conjugate wherein the oligonucleotide is a polynucleotide in the form of an expression cassette suitable for gene therapy is another embodiment of the invention. Further embodiments are conjugates wherein the oligonucleotide, oligonucleotide equivalent or polynucleotide is non-covalently conjugated to the glycoside via a cationic entity that complexes nucleic acids.

In an embodiment said glycoside of the conjugate of the invention is a mono-, di- or tri-saccharide, or any higher order saccharide, and wherein said saccharide comprises at least one mannose-6-phosphate residue. In a further embodiment said saccharide comprises at least two mannose-6-phosphate residues. In yet a further embodiment said di-, tri- or higher order saccharides are linked via (α1,2), (α1,3) or (α1,6) linkages. In yet another embodiment said glycoside is a bi-antennary or tri-antennary oligosaccharide comprising mono-, di- or tri-saccharides or any higher order saccharides, wherein said saccharides comprise at least one mannose-6-phosphate residue, preferably said saccharides comprise at least two mannose-6-phosphate residues.

In a further embodiment said compound of the conjugate of the invention is a growth factor, a vaccine, a vitamin, an antibody or a cationic entity to complex nucleic acids, in particular PEI. Also said compound of the conjugate of the invention can be any moiety that is functional or can be functionalized in the nucleus of a cell, in particular a muscle cell.

In yet a further embodiment said glycoside is linked to said compound, in particular an oligonucleotide or oligonucleotide equivalent, via a labile spacer that can be cleaved intracellularly.

In another embodiment the invention relates to a method for producing a glycoside-compound conjugate, characterised by linking at least one glycoside comprising at least one mannose-6-phosphate residue with an oligonucleotide selected from any one of the following: DNA, RNA, PNA, LNA, morpholino, 2′-O-methyl RNA, or 2′-O-allyl RNA.

Delivery in the Nucleus

The M6P targeting system is meant for import into the lysosomal compartment of cells and GAA can only exert its effect in the lysosomes where it must, and does in a therapeutic setting, hydrolyse glycogen causing the disease.

The exon splicing process takes place in the nucleus and certainly not in the lysosomes where there is no RNA to be spliced. The surprising discovery the inventor made is that M6P when coupled to an antisense molecule complementary to a splice site can also direct its cargo to the splicing machinery which is at a location distinctly different from the “well-known destination” normally used by M6P and its cargo (GAA). This unexpected discovery made it possible for the inventor to use M6P to target muscle cells with bioactive compounds to various cellular compartments such as the nucleus (as an unexpected result, since the M6P targeting system is believed to direct cargo to the lysosomal compartment).

Thus the invention further concerns the use of any of the glycoside-compound conjugates of the invention to alter the sequence of an RNA or its precursors, to modify or modulate its composition and arrangement of its exons such that a protein can be made able to restore functionality of a cell to which it is delivered, in particular of muscle cells. In one aspect the glycoside-compound conjugates of the invention may be used to block or stimulate any RNA that can lead to improved performance of heart, respiratory or skeletal muscles with the aim to ameliorate the progression of certain diseases or impairments associated with e.g. ageing.

In another aspect the invention relates to a method for delivering an oligonucleotide or oligonucleotide equivalent into the nucleus of cells comprising an insulin-like growth factor II/mannose-6-phosphate receptor (IGF-II/MPR), characterized by contacting a glycoside-oligonucleotide conjugate, wherein said glycoside is a ligand capable of binding to a mannose-6-phosphate receptor with said cells. In one embodiment of the method of the invention said oligonucleotide or oligonucleotide equivalent is selected from the group consisting of RNA, DNA, morpholino, 2′-O-methyl RNA, or 2′-O-allyl RNA, Peptide Nucleic Acid (PNA) and Locked Nucleic Acid (LNA). In a further embodiment said oligonucleotide or oligonucleotide equivalent comprises at least 10 nucleotides identical or complementary to a human dystrophin gene. In yet a further embodiment said glycoside is a mono-, di- or tri-saccharide, or any higher order saccharide, and wherein said saccharide comprises at least one mannose-6-phosphate residue. In yet a further embodiment said glycoside is selected from the group consisting of a bi-antennary, a tri-antennary and a tetra-antennary oligosaccharide comprising mono-, di- or tri-saccharides or any higher order saccharides, wherein said saccharides comprise at least one mannose-6-phosphate residue. In another embodiment said glycoside is linked to said oligonucleotide or oligonucleotide equivalent via a labile spacer that can be cleaved intracellularly.

In an embodiment of the method of the invention said cells are muscle cells of a patient selected from the group consisting of Duchenne Muscular Dystrophy, Beckers Muscular Dystrophy, spinal muscular atrophy (SMA), bethlem myopathy, myotubular myopathy, limb-girdle muscular dystrophy 2A and 2B, Miyoshi myopathy, myotonic dystrophy, lysosomal storage disorders and merosin deficient muscular dystrophy, and said contacting of said glycoside-oligonucleotide conjugate with said cells is by administration to said patient of a therapeutically effective amount of said glycoside-oligonucleotide conjugate together with a pharmaceutically acceptable carrier and said method thus relates to the treatment of muscle diseases. In a particular embodiment said cells are muscle cells of a Duchenne Muscular Dystrophy (DMD) patient and wherein said oligonucleotide or oligonucleotide equivalent is an antisense oligonucleotide which causes exon skipping and induces or restores the synthesis of dystrophin or variants thereof. In an embodiment said contacting comprises injection into animal or human tissue.

Further the invention relates to a method for inducing the synthesis or functioning of any RNA species in muscle cells, in which said cells are contacted with a glycoside-oligonucleotide conjugate of the invention, whereby said oligonucleotide inhibits or reduces the activity of RNAs or proteins repressing the synthesis or functioning of said RNA species.

Further the invention relates to a method for inhibiting or reducing the synthesis or functioning of any RNA species in muscle cells which causes disease or predisposition of disease, which may be of viral or bacterial origin, in which said muscle cells are contacted with a glycoside-oligonucleotide conjugate according to the invention, whereby said oligonucleotide inhibits the synthesis or functioning of said RNA species.

Also the present method is applicable in gene therapy which in other words means that the invention also relates to a method for delivering an oligonucleotide into the nucleus of cells comprising an insulin-like growth factor II/mannose-6-phosphate receptor (IGF-II/MPR), in particular muscle cells, wherein said oligonucleotide is a polynucleotide which induces the synthesis or functioning of RNAs or proteins in muscle cells thereby alleviating diseases or predisposition of disease, wherein said method comprises contacting a glycoside-polynucleotide conjugate, wherein said glycoside is a ligand capable of binding to a mannose-6-phosphate receptor with said cells. Such a polynucleotide may thus be an expression cassette suitable for gene therapy. In an embodiment the polynucleotide is non-covalently conjugated to the glycoside via a cationic entity that complexes nucleic acids. Also in the method of the invention the oligonucleotide or mucleotide equivalent may be non-covalently conjugated to the glycoside via a cationic entity that complexes nucleic acids. A cationic entity that complexes nucleic acids is PEI.

Further the glycoside-oligonucleotide conjugates of the invention may be of use to increase the body muscle mass of farm animals. For instance muscle cells may be targeted with a conjugate comprising a compound which is designed to increase muscle mass, such as for instance an oligonucleotide that inhibits myostatin production. Accordingly the invention also relates to such a method.

Further the invention relates to a method for delivering a vaccine into muscle cells, in which muscle cells are contacted with a glycoside-compound conjugate according to the invention, wherein said compound is a vaccine, in particular a DNA vaccine.

Further the invention relates to the use of a glycoside-compound conjugate according to the invention in the therapeutic treatment of muscle diseases. In particular the invention relates to the use of a glycoside-compound conjugate according to the invention for the preparation of a medicament. In an embodiment the invention relates to the use of a glycoside-compound conjugate according to the invention for the preparation of a medicament for the therapeutic treatment of muscle diseases.

In a further embodiment the glycoside-compound conjugate, in particular the glycoside-oligonucleotide conjugate, further comprises a marker. In an embodiment said marker is directly or indirectly detectable by visual, chemical or molecular methods. In an embodiment said marker is a fluorescent marker, a chemiluminescent marker, a radioactive marker, an enzymatic marker or molecular marker.

All references, including all patents and other publications identified herein, are incorporated herein by reference in their entireties for the purpose of describing and disclosing, for example, information that may be used in connection with the present invention. These publications are provided solely for their disclosure prior to the filing date of the present application.

Further the invention relates to a method for in vivo or in vitro diagnostic tests, in which a conjugate of the invention further comprising a marker is contacted with muscle cells and detecting directly or indirectly the presence or absence of said marker. Yet further the invention concerns a diagnostic detection kit comprising a conjugate of the invention further comprising a marker and optionally further comprising detection reagents.

### EXAMPLES

### Example 1

### Overview Building Blocks for Synthesis of the Glycoside-Compounds

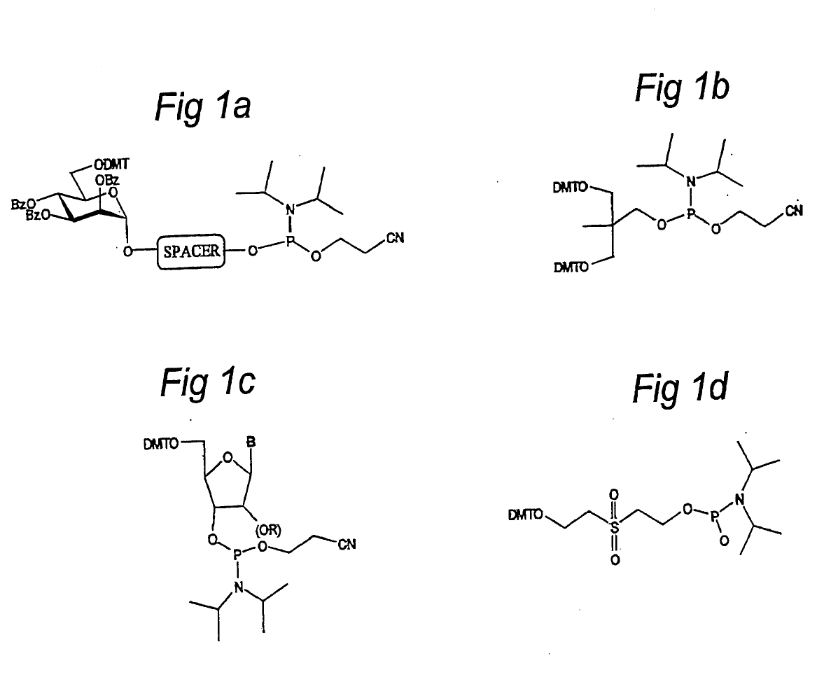

To be able to produce the glycoside-compounds, a multiple step synthesis was designed. All syntheses were performed using standards organic chemical synthesis procedures. The separate building blocks 1A and 1B (FIGS. 1A and 1B respectively) were synthesised, whereas the remaining blocks (FIGS. 1C and 1D) were purchased (FIG. 1).

### Example 2

### Assembly of Building Block 1

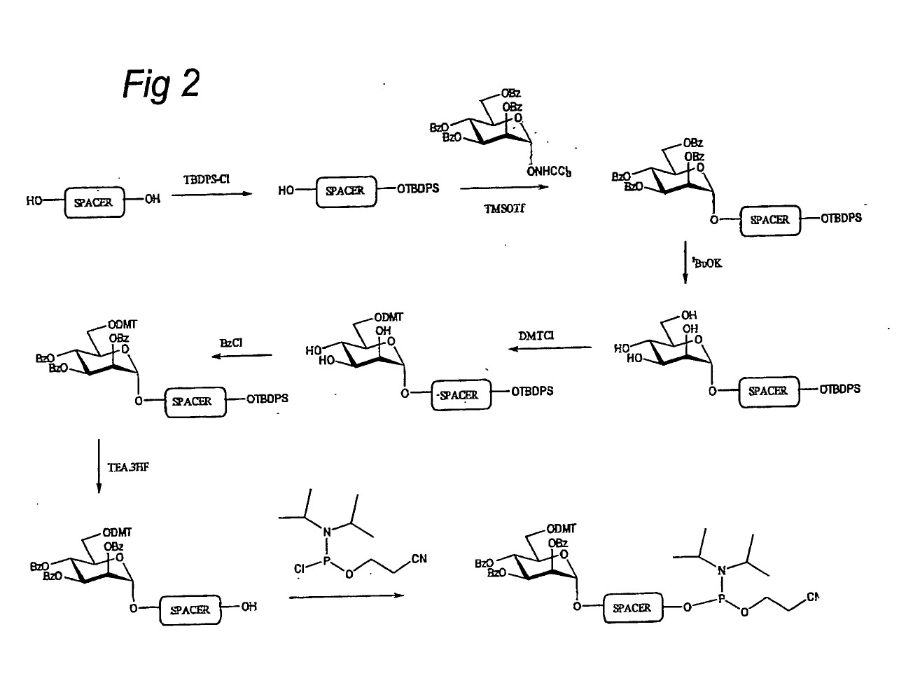

Building block 1 (FIG. 2) is composed of the glycoside linked through a SPACER to a moiety X. SPACER is composed of a C4-, C5-, or C11-alkyl or tetrathylene glycol. Moiety X is composed of a phosphate, amide or disulfide bond.

### Example 3

### Assembly of Building Block 2

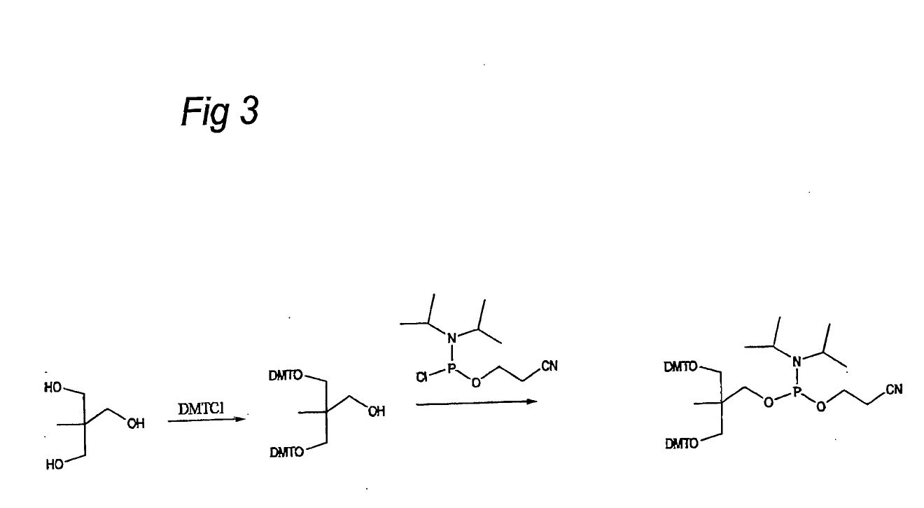

Building block 2 (FIG. 3) is designed to connect Building block 1 to the compound, in example 4 to an oligonucleotide.

### Example 4

### Assembly of the (man-6P)₂-en (man-6P)₄-oligonucleotides with C₄-, C₅-, and C₁₁-alkyl and Tetraethylene Glycol Spacers

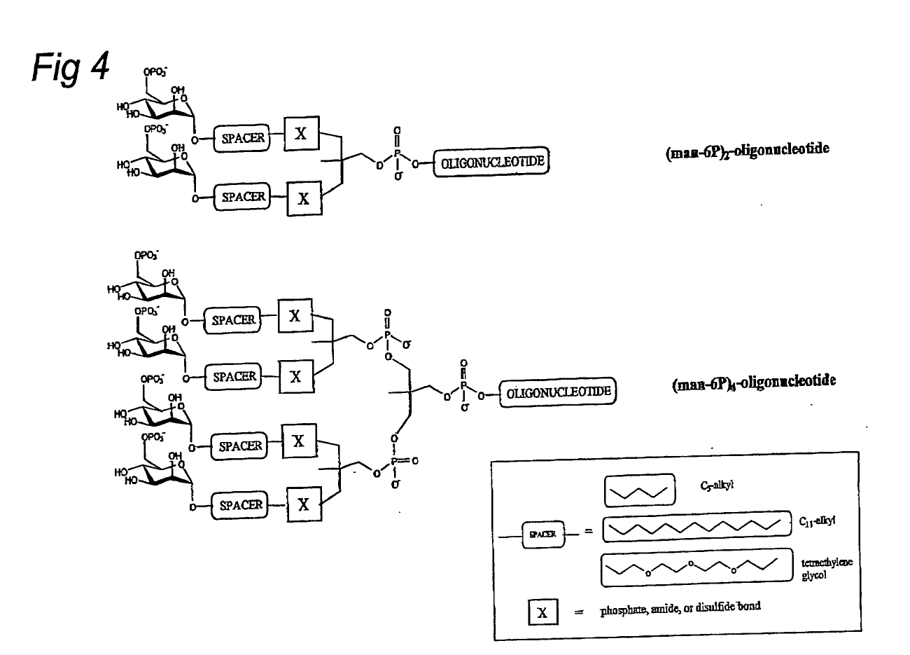

Using standard amidite solid phase synthesis the (man-6)ₓ-oligonucleotides were synthesized (FIG. 4).

### Example 5

### Uptake of the (man-6P)₂- and (man-6P)₄-oligonucleotides by Muscle Cells

A) Using standard molecular biological techniques, the di-antennary ((man-6P)₂) and tetra-antennary ((man-6P)₄)-monosaccharide-oligonucleotide conjugates (as described in example 4) were end-labelled with fluorescein. C2C12 cells (murine muscle cells) were grown to confluency and allowed to differentiate into multi-nucleated myotubes (i.e. structures resembling mature muscle fibers) by incubation in low-serum medium for 7 to 14 days. The fluorescent compounds were added to the cells in one ml medium, and, after 4 hours of incubation at 37° C., the cells were inspected for uptake. The results indicate that the compounds were indeed taken up efficiently.

B) In a similar manner as in example 5A) it was shown that KM109 cells (primary human muscle cells) efficiently take up ((man-6P)₂) and tetra-antennary ((man-6P)₄)-monosaccharide-oligonucleotide conjugates.

### REFERENCES

Aartsma-Rus A., Bremmer-Bout M., Janson A. A. M., den Dunnen J. T., van Ommen G. J. B. and van Deutekom J. C. T. (2002) Targeted exon skipping as a potential gene correction therapy for Duchenne muscular dystrophy. Neuromuscul. Disord. 12, S71-S77.
Bijvoet A. G. A., Kroos M. A., Pieper F. R., Van der Vliet M., de Boer H. A., Van der Ploeg A. T., Verbeet M. P. and Reuser A. J. (1998) Recombinant human acid α-glucosidase: high-level production in mouse milk, biochemical characteristics, correction of enzyme deficiency in GSDII KO mice. Hum. Mol. Genet. 7, 1815-1824.
Dahms N. M. and Hancock M. K. (2002) P-type lectins. Biochim. Biophys. Acta 1572, 317-340.
Funk B., Kessler U., Eisenmenger W., Hansmann A., Kolb H. J. and Kiess W. (1992) Expression of the insulin-like growth factor-II/mannose-6-phosphate receptor in multiple human tissues during fetal life and early infancy. J. Clin. Endocrinol. Metab. 75, 424-431.
Ghosh P., Dahms N. M. and Kornfeld S. (2003) Mannose 6-phosphate receptors: new twists in the tale. Nat. Rev. Mol. Cell Biol. 4, 202-212.
Gollins H., McMahon J., Wells K. E. and Wells D. J. (2003) High-efficiency plasmid gene transfer into dystrophic muscle. Gene Ther. 10, 504-512.
Hassan A. B. (2003) Keys to the hidden treasures of the mannose 6-phosphate/insulin-like growth factor 2 receptor. Am. J. Pathol. 162, 3-6.
Herweijer H., Whitesell L. F., Buck J., Bates M. K., Budker V., Zhang G., Wolff J. A. and Wolff M. R. (2000) Retrograde coronary venous delivery of naked plasmid DNA. Mol. Ther. 1, S202.
Herweijer H. and Wolff J. A. (2003) Progress and prospects: naked DNA gene transfer and therapy. Gene Ther. 10, 453-458.
Lu Q. L., Liang H.-D., Partridge T. and Blomley M. J. K. (2003) Microbubble ultrasound improves the efficiency of gene transduction in skeletal muscle in vivo with reduced tissue damage. Gene Ther. 10, 396-405.
Martiniuk F., Chen A., Donnabella V., Arvanitopoulos E., Slonim A. E., Raben N., Plotz P. and Rom W. N. (2000) Correction of glycogen storage disease type II by enzyme replacement with a recombinant human acid maltase produced by over-expression in a CHO-DHFR^{neg} cell line. Biochem. Biophys. Res. Commun. 276, 917-923.
Reuser A. J. J., Kroos M. A., Ponne N. J., Wolterman R. A., Loonen M. C. B., Busch H. F. M., Visser W. J. and Bolhuis P. A. (1984) Uptake and stability of human and bovine acid α-glucosidase in cultured fibroblasts and skeletal muscle cells from glycogenosis type II patients. Exp. Cell Res. 155, 178-189.
Samoylova T. I. and Smith B. F. (1999) Elucidation of muscle-binding peptides by phage display screening. Muscle Nerve 22, 460-466.
van Deutekom J. C. T., Bremmer-Bout M., Janson A. A. M., Ginjaar I. B., Baas F., den Dunnen J. T. and van Ommen G. J. B. (2001) Antisense-induced exon skipping restores dystrophin expression in DMD patient derived muscle cells. Hum. Mol. Genet. 10, 1547-1554.
Weisbart R. H., Yang F., Chan G., Wakelin R., Ferreri K., Zack D. J., Harrison B., Leinwand L. A. and Cole G. M. (2003) Cell type specific targeted intracellular delivery into muscle of a monoclonal antibody that binds myosin IIb. Mol. Immunol. 39, 783-789.
Wenk J., Hille A. and von Figura K. (1991) Quantitation of Mr 46000 and Mr 300000 mannose-6-phosphate receptors in human cells and tissues. Biochem. Int. 23, 723-731.
Zhang G., Budker V., Williams P., Subbotin V. and Wolff J. A. (2001) Efficient expression of naked DNA delivered intra-arterially to limb muscles of nonhuman primates. Hum. Gene Ther. 12, 427-438.

What I claim is:

1. A method for delivering an oligonucleotide or oligonucleotide equivalent into the nucleus of cells comprising an insulin-like growth factor II/mannose-6-phosphate receptor (IGF-II/MPR), said method comprising contacting a glycoside-oligonucleotide conjugate, wherein said glycoside is a ligand capable of binding to a mannose-6-phosphate receptor with said cells.

2. The method according to claim 1, wherein said oligonucleotide or oligonucleotide equivalent is selected from the group consisting of RNA, DNA, morpholino, 2′-O-methyl RNA, or 2′-O-allyl RNA, Peptide Nucleic Acid (PNA) and Locked Nucleic Acid (LNA).

3. The method according to claim 2, wherein said oligonucleotide or oligonucleotide equivalent comprises a length of at least 10 nucleotides identical or complementary to a human dystrophin gene.

4. The method according to claim 1, wherein said glycoside is a mono-, di- or tri-saccharide, or any higher order saccharide, and wherein said saccharide comprises at least one mannose-6-phosphate residue.

5. The method according to claim 1, wherein said glycoside is selected from the group consisting of a bi-antennary, a tri-antennary and a tetra-antennary oligosaccharide comprising mono-, di- or tri-saccharides or any higher order saccharides, wherein said saccharides comprise at least one mannose-6-phosphate residue.

6. The method according to claim 1, wherein said glycoside is linked to said oligonucleotide or oligonucleotide equivalent via a labile spacer that can be cleaved intracellularly.

7. The method according to claim 1 wherein said cells are muscle cells of a patient selected from the group consisting of Duchenne Muscular Dystrophy, Beckers Muscular Dystrophy, spinal muscular atrophy (SMA), bethlem myopathy, myotubular myopathy, limb-girdle muscular dystrophy 2A and 2B, Miyoshi myopathy, myotonic dystrophy, lysosomal storage disorders and merosin deficient muscular dystrophy, and said contacting of said glycoside-oligonucleotide conjugate with said cells is by administration to said patient of a therapeutically effective amount of said glycoside-oligonucleotide conjugate together with a pharmaceutically acceptable carrier.

8. The method according to claim 1, wherein said cells are muscle cells of a Duchenne Muscular Dystrophy (DMD) patient and wherein said oligonucleotide or oligonucleotide equivalent is an antisense oligonucleotide which causes exon skipping and induces or restores the synthesis of dystrophin or variants thereof.

9. The method according to claim 1 wherein said oligonucleotide or oligonucleotide equivalent induces the synthesis or functioning of any RNA species in muscle cells, by inhibiting or reducing the activity of RNAs or proteins repressing the synthesis or functioning of said RNA species.

10. The method according to claim 1 wherein said oligonucleotide or oligonucleotide equivalent reduces the synthesis or functioning of any RNA species in muscle cells which causes disease or predisposition of disease, whereby said oligonucleotide inhibits the synthesis or functioning of said RNA species.

11. The method according to claim 1 wherein said oligonucleotide is a polynucleotide which induces the synthesis or functioning of RNAs or proteins in muscle cells thereby alleviating diseases or predisposition of disease.

12. The method according to claim 11 wherein the polynucleotide is non-covalently conjugated to the glycoside via a cationic entity that complexes nucleic acids.

13. The method according to claim 1 wherein the glycoside-oligonucleotide conjugate further comprises a marker.

14. The method according to claim 13 for in vivo or in vitro diagnostic tests said method further comprising detecting directly or indirectly the presence or absence of said marker.

15. The method according to claim 1 wherein the oligonucleotide is a vaccin.

16. A conjugate comprising a glycoside linked to an oligonucleotide or oligonucleotide equivalent, said glycoside being a ligand capable of binding to a mannose-6-phosphate receptor of a muscle cell and said oligonucleotide or oligonucleotide equivalent comprising at least 10 nucleotides identical or complementary to a human dystrophin gene.

17. The conjugate according to claim 16, wherein said oligonucleotide or oligonucleotide equivalent is selected from the group consisting of RNA, DNA, morpholino, 2′-O-methyl RNA, or 2′-O-allyl RNA, Peptide Nucleic Acid (PNA) and Locked Nucleic Acid (LNA).

18. The conjugate according to claim 16, wherein said glycoside is a mono-, di- or tri-saccharide, or any higher order saccharide, and wherein said saccharide comprises at least one mannose-6-phosphate residue.

19. The conjugate according to claim 16, wherein said glycoside is a bi-antennary or tri-antennary oligosaccharide comprising mono-, di- or tri-saccharides or any higher order saccharides, wherein said saccharides comprise at least one mannose-6-phosphate residue.

20. The conjugate according to claim 16, wherein said glycoside is linked to said oligonucleotide or oligonucleotide equivalent via a labile spacer that can be cleaved intracellularly.

21. The conjugate according to claim 16 wherein the oligonucleotide is a polynucleotide in the form of an expression cassette suitable for gene therapy.

22. The conjugate according to claim 21 wherein the polynucleotide is non-covalently conjugated to the glycoside via a cationic entity that complexes nucleic acids.
